(19) United States
(12) Patent Application Publication
(10) Pub. No.: US 2021/0052167 A1
(43) Pub. Date: Feb. 25, 2021

(54) METHOD FOR MONITORING CARDIOVASCULAR FUNCTIONS AND PORTABLE EQUIPMENT IMPLEMENTING THE METHOD

(71) Applicant: UAB CAROMON, Vilnius (LT)

(72) Inventor: Albinas STANKUS, Kaunas (LT)

(21) Appl. No.: 16/979,039

(22) PCT Filed: Feb. 7, 2019
(86) PCT No.: PCT/IB2019/050978

§ 371 Date: Sep. 8, 2020

(30) Foreign Application Priority Data
Mar. 5, 2018 (LT) LT2018508

(51) Int. Cl. A61B 5/0205; A61B 5/02; A61B 5/0456; A61B 5/00

(52) U.S. Cl. CPC A61B 5/0205; A61B 5/02007; A61B 5/0456; A61B 5/02116; A61B 5/6817; A61B 5/6816; A61B 5/7203

(57) ABSTRACT

The invention provides a new method for monitoring and analysing cardiovascular functions and equipment implementing the method. The portable equipment described and measurement analysis method operating in it, allowing continuous monitoring over time of cardiac functions and vascular resistance by electrical impedance and mechanical methods with electrocardiogram (ECG) by assessing changes in the carotid pulse wave form, carotid wave amplitude level, cardiac contraction, heart rate variability. A new device (a piece of equipment described), a part of which is fitted in outer ear canals and is designed for measuring the electrical head tissue impedance and ear air pressure parameters. The method and equipment provide the possibility to measure, process, synchronize and analyse cardiovascular activity parameters of three types and thereby obtain information about the cardiovascular activity status and its variation over time, which could not be obtained by measuring and analysing these parameters separately.

### FIELD OF THE INVENTION

The invention relates the field of medical equipment, and in particular, it is a portable equipment and measurement analysis method operating in it, allowing the continuous monitoring over time of cardiovascular functions by electrical impedance and mechanical methods with electrocardiogram (ECG).

### THE RELATED ART

This invention provides a method and technical equipment implementing the method for monitoring parameters of the human cardiac activity and vascular system. The invention is novel in that three parameters are measured: electrical head tissue impedance, mechanical air pressure in cars caused by the circulatory activity and electrocardiogram. All measurements are performed over time and synchronized according the electrocardiogram R-peak. Measurements from different sources are unified and analysed together, so the data measured in other sources supplements, validate the data of one type measured, provides additional information, provides a possibility to use a principle of combining different measurements when analysing data and to get more information by combining than by analysing each different measurement separately, not synchronized.

The equipment provided for implementing the measurement method, and a new piece of equipment, a device for measuring the electrical head tissue impedance and the air pressure in ears.

Document U.S. Pat. No. 8,211,031 B2 (published on 3 Jul. 2012) discloses a device and method for measuring cardiovascular parameters which measures the electrical head impedance. The results of the electrical impedance measurement alone do not provide the possibility to measure other parameters of the cardiac and circulatory system, there is no possibility to synchronize different measurements, to obtain data from said synchronization, which cannot be obtained by measuring the electrical impedance alone.

Document US20110190600A 1 (published on 4 Aug. 2011) provides a physiological sensor system and measurement method using those sensors. The cited document lists many sensors (electrodes, optical detectors, temperature sensors, etc.), provides a measurement method with those sensors, but nothing is mentioned about the processing of the measured parameters, synchronization, performance of the required analysis, the description does not clearly specify the way many of the listed sensors can be linked to the system to achieve a particular analysis result, a principle of linking the data measured by different sensors is not provided.

The presented solutions of the related art are characterized by the following deficiencies:
only the electrical tissue impedance is measured, it is not taken into account, is not comparable to other cardiovascular system parameters, i.e. it is lack of versatile data and holistic analysis;
the electrical measurement alone does not allow the measurement of mechanical, pressure blood flow parameters;
the system with a plurality of physiological sensors is provided, but the calculation method is not described, it is not clear what analysis can be performed.

This invention provides a technical solution that does not have the above deficiencies.

### SUMMARY OF THE INVENTION

The invention provides a new method for monitoring and analysing cardiovascular functions and equipment implementing the method. The portable equipment and measurement analysis method operating in it, allowing continuous monitoring over time of cardiac functions and vascular resistance by electrical impedance and mechanical methods with electrocardiogram (ECG) by assessing changes in the carotid pulse wave form, carotid wave amplitude, cardiac contraction, heart rate variability, is described. There is also a new device (a piece of equipment described), a part of which is fitted into outer ear canals and is designed for measuring the electrical head tissue impedance and parameters of the air pressure in ears.

The method and equipment make it possible to measure, process, synchronize, analyse cardiovascular parameters of four types, and thus obtaining information about the cardiovascular status and its change over time, which could not be obtained by measuring and analysing said parameters separately.

### BRIEF DESCRIPTION OF DRAWINGS

FIG. 1 depicts a structural diagram of the whole equipment provided by this invention. Numbers marked in FIG. 1:

1.1 air pressure sensor for mechanical measurement method, measuring air pressure in the ear;

1.2 pressure sensor signal amplifier;

1.3 power supply for electrical impedance tissue resistance measurement;

1.4 high frequency amplifier for electrical impedance tissue resistance measurement;

1.5 variable part detector;

1.6 low frequency amplifier;

1.7 fixed part detector;

1.8 analog to digital signal converter;

1.9 ECG signal amplifier;

1.10 processing of the ECG R-peak trigger information;

1.11 ECG R-peak detector;

1.12 microcontroller;

1.13 power supply;

1.14 data block;

1.15 algorithms, methods used in the analysis;

1.16 equipment for sending and receiving data;

1.17 distant cloud server;

1.18 database;

1.19 printer or other information display device;

1.20 computer;

1.21 portable device;

1.22 analysis results.

FIG. 2 depicts a stethoscope-like measuring device for measuring the air pressure.

FIG. 3 depicts a structural diagram of the ear plug of the measuring device. At the bottom—side view, top view at the top.

FIG. 4, FIG. 5 and FIG. 6 depict characteristic points of characteristic parameters of measurements and measurements analysed.

The presented figures are more illustrative, scale, proportions and other aspects do not necessarily correspond to a real technical solution.

### DETAILED DESCRIPTION

The invention provides a method and equipment implementing the method for determining the human heart blood ejection force, the arterial filling and stiffness value, and heart rate variability.

The pulse pressure wave (PPW) is the sum of the wave created by the heart and its reflections. Currently, there is no unified, comprehensive, universal PPW analysis method. Different forms of the PPW am obtained by registering the PPW at various points in the human arterial system. The PPW form is determined by the cardiac inotropic function and vascular resistance, these phenomena are reflected in the form of the PPW. Due to its proximity to the aorta, this is most noticeable in the carotid wave form. Pulse waves are divided into three classes based on their duration and the “secondary” wave caused by reflections in the arterial blood pressure aortic bow. Type A wave is characterized by that an early systolic wave (ESW) is lower than a late systolic wave (LSW). Conversely, for type C waves, the ESW is higher than the LSW. Intermediate waves are assigned to type B.

The carotid ESW is associated with the cardiac ejection fraction, its contraction and initial filling, and the LSW indicates that returning previous or later wave reflection has full information about the entire vascular tract and depends on the size of the reflected wave, pulse wave velocity, arterial blood pressure, vascular stiffness and many other properties.

The present description provides an invention which used under determined carotid pulse wave form can explain changes that have occurred and occur in systemic circulatory, more specifically, characterize the cardiac activity and central circulatory system resistance by using direct simple measurements, rather than complex analyses and calculations.

The invention provides a portable equipment and measurement analysis method that allows electrocardiogram (ECG) to continuously monitor cardiac functions and vascular resistance over time by electrical impedance and mechanical methods by assessing changes in the carotid pulse wave form, carotid wave amplitude, cardiac contraction phases, heart rate variability in calmness and being affected by medicines and/or various other factors.

As mentioned, in the present invention, the following features are measured to determine cardiovascular parameters and their change over time:

the PPW by the electrical impedance method

the PPW by the mechanical method by measuring air pressure

by electrocardiogram (ECG).

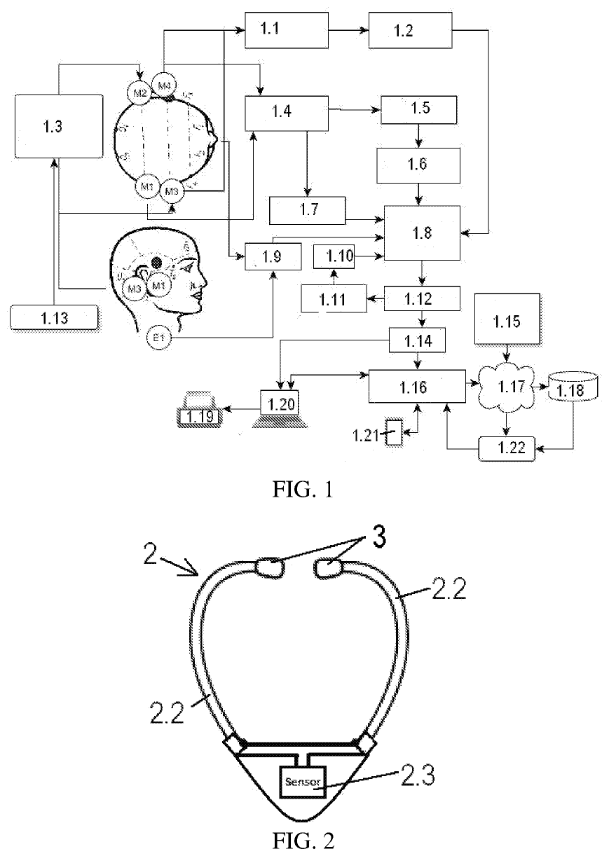

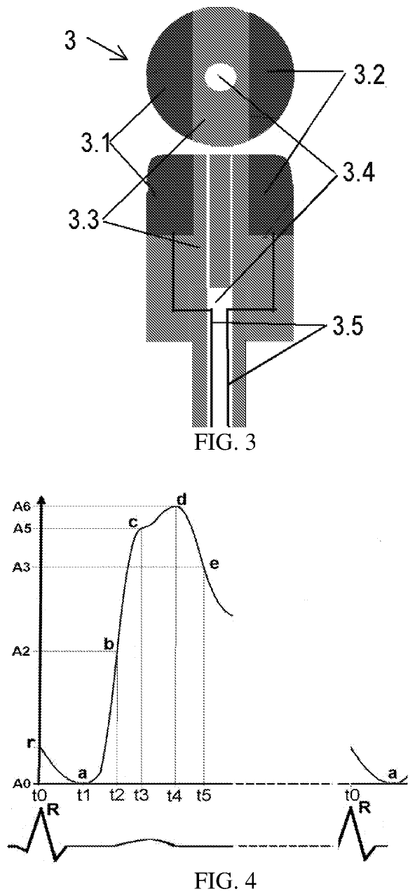

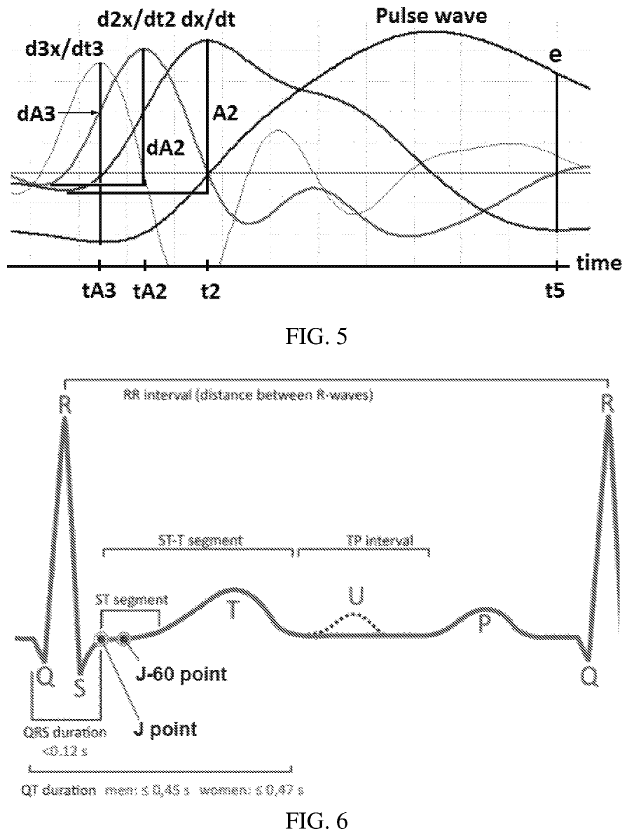

All the data measured are processed, analysed, compared and presented. FIG. 1 is a schematic diagram of the data measurement, processing, analysis and presentation equipment.

The carotid PPW is measured by two methods: by electrical impedance and mechanical methods. The electrical impedance PPW measurement method is used to register volume changes in large arteries, because the relative impedance change is proportional to the relative volume change: AZ/Z=k*AV/V, where DZ is the variable impedance part value, Z is the fixed impedance part value, AV is the volume change. Since the distance between electrodes is fixed, this equation can be transformed into AZ/Z=k*AS/S, where S is the total area of the blood vessels, and k is the proportionality factor, the value of which depends on the distance between electrodes and the electrical tissue conductivity between electrodes.

The present invention provides a device (2), which is a part of the measurement equipment, measuring the PPW by the electrical impedance method. The electrical impedance measurement method is based on the electrical tissue resistance measurement. In order to measure the electrical tissue resistance on different sides of the measured object (in the present invention, of the head) the pair of electrodes is arranged: one electrode of the pair (current electrode) generates the current of determined parameters (0.1-0.9 mA, 20-100 kHz), the current generated passes through measured tissues and enters the other (measurement) electrode on the other side of the measured object. The electrical current parameters measured by the measurement electrode are designed for determining the electrical tissue resistance, and the electrical resistance, as mentioned above, is proportional to the area of the blood vessels.

The device (2) (FIG. 2) used in the present invention resembles in its shape a stethoscope having two ends, plugs (3) (FIG. 3) fitted and easily pressed into human ears, into outer ear canals. As usual, said plugs (3) fitted into ears resemble in its shape a cylinder with rounded edges. Plugs (3) of the device fitted into ears have said electrical impedance measurement electrodes. One of possible arrangements of electrodes is when the current electrode (3.1) is arranged on one side of the cylindrical plug (3) and the other (measurement) electrode (3.2) is arranged on the other (opposite) side of the plug (3) and between these current (3.1) and measurement (3.2) electrodes, i.e. electrodes are separated from each other by an electrically non-conductive material (3.3), thus electrodes are electrically isolated from each other. Alternatively, the pair of current (3.1) and measurement (3.2) electrodes can be assembled into plugs (3) fitted into ears. It is important that electrodes (3.1) and (3.2) are separated from each other by the electrically non-conductive material (3.3). Electrodes (3.1) and (3.2) must be comfortable to fit into the ear, and good electrical contact with ear tissues which are in contact with electrodes (3.1) and (3.2) is required. The current electrode (3.1) in the plug (3) is electrically connected to the current generating device of determined parameters using electrically conductive electrical transmission means (3.5); the measurement electrode (3.2) is electrically connected to the measurement data processing device.

Other embodiment of the same invention is possible, when measuring cardiovascular activity parameters by the electrical impedance method, a device with two pairs of electrodes (a pair: current electrode and measurement electrode) is used. The device embraces mechanically and presses electrodes on one side of the earlobe. The current electrode is pressed on one side of the earlobe, and the measurement electrode is pressed on the other (opposite) side of the earlobe.

Other embodiment of the same invention is possible, when measuring cardiovascular activity parameters by the electrical impedance method, conventional, electrically conductive, flat electrodes are fixed at measurement points on the skin behind the ear at nipple (mastoid) bones. In this case, a pair of the current electrode and measurement electrode is attached on one side of the head and another pair of electrodes is attached on the other side of the head. In any of the embodiments of the present invention, electrodes are fixed at the point of the head where the venous circulatory, breathing effect is at least expressed, and major carotid branches are mostly expressed.

The mechanical PPW measurement is based on changes in blood pressure on blood vessel walls in a closed cavity, which consists of volumes of the ear and measured parts. Due to arterial tonic and pulse blood flow, the volume of fine arterioles and capillaries in the skin of the ear changes, resulting in additional and dynamic air pressure. The measured pressure consists of fixed and variable parts. The variable part represents the carotid PPW form, which is analysed by analogy as measured by the electrical impedance method. The fixed part depends on the existing autonomic nervous regulation of arterioles, which is proportional to the regulation of the blood vessels in the brain. The condition of the latter varies depending on the condition of the subject. Enlargement (dilation) of the blood vessels in the brain and narrowing of peripheral blood vessels have been observed in response to various stimuli in the body, such as sound, light, electrical or thermal stimulation. Irritants cause a reference reflex. The reference reflex is separated from the defensive reflex when the cerebral blood vessels narrow (spasm). The increase of the fixed part indicates the expansion of blood vessels of the head and the decrease is the narrowing.

In the present invention, the same device (2) is used for mechanical measurement of the PPW, as in the case of the electrical PPW impedance measurement, by adding additional elements for mechanical PPW measurement. The device (2) resembles in its shape a stethoscope as mentioned above, plugs (3) fitted into ears are electrodes (3.1) and (3.2) separated by an electrically insulating material (3.3). The said electrically insulating material (3.3) comprises a hollow tube, an air channel (3.4). Alternatively, the hollow tube, air channel (3.4) can be assembled in ear plugs (3). The said hollow tube, air channel (3.4) extends along the longitudinal portion (body) of the stethoscope-like device (2), which, at the other end than the plug (3) in the ear, holds an air pressure sensor (2.3). In a closed cavity the air pressure sensor (2.3) transforms the air pressure parameters, the air pressure changes the wave into the electrical signal of the respective parameters. The above-mentioned air channel (3.4) extends from both ear plugs (3) through the elongated part (housing) of the stethoscope-like device (2) to the point where air channels (3.4) coming from both ear plugs (3) are connected and an air pressure sensor (2.3), which measures changes in the air pressure generated by the circulatory in ears, is arranged in the connection. The air pressure sensor (2.3) converts the air pressure to the electrical signal of determined parameters, which is further transmitted for processing and analysis.

In the situation described above, when one air pressure sensor (2.3) measures changes in the air pressure of both ears, there is a problem if one wants to measure the pressure of each ear separately or parameters of the pressure of one ear and the other ear. For such measurements in air channels (3.4), which connect ear plugs (3) with the pressure measuring sensor (2.3), the air flow limitation technical devices, such as valves preventing air movement in the air channels, are installed. These air valves may allow air to flow from one (any) ear plug (3) to the sensor (2.3), from both plugs (3) to the sensor (2.3), or to prevent air flow from plugs (3) to the sensor (2.3).

In other embodiment of the invention, the air pressure sensor (2.3) is arranged in said plug (3) in the ear to prevent the transmission of the air pressure wave in the housing by the hollow tube (3.4) to the pressure sensor (2.3), to provide a possibility to measure the pressure of each car separately and other functions. One air pressure sensor (2.3) is arranged in the ear plug (3), thus measuring the air pressure and converting the measured pressure parameters into electrical signals of determined parameters in each ear plug (3), measured electrical signals are transmitted for processing and analysis. In this way, it is possible to measure the air pressure of only one selected ear, and the possibility to synchronize the air pressure parameters of different ears appears.

II derivative is chosen for electrocardiogram (ECG) registration. One ECG measurement electrode coincides with the right electrical ear impedance measuring device measurement electrode, the second electrode is arranged on the left leg. The signal from the ECG electrodes is processed and submitted for sampling, processing after which the ECG R rise or peak is distinguished and the trigger signal (Trig) is prepared for the synchronization of other measured parameters according to the distinguished ECG R peak.

As mentioned, the results of electrical impedance and mechanical measurements are synchronized according to ECG R peak, i.e. parameters measured by different methods are submitted for further data processing and displaying after synchronization over time according to ECG R peak. One of the measurement data processing is data sampling. In the present invention, at least two methods of sampling are possible: by selecting (initially −450 ms) and continuously recalculating the duration of the sampling period or performing the sampling continuously. After each heartbeat, four measurement data blocks are obtained: the electrical carotid impedance measurement variable part (E_block) and the fixed part (P_block), the mechanical measurement block (M_block) and the ECG (R_block) which with tagged ECG R peak are sent in real time for further data processing and analysis.

The data processing method of the present invention has the following steps:

1. The noise is eliminated in four processes by filters without a phase shift (filtering twice up and down).

2. Four process blocks are obtained during registration: ECG (R_block), electrical impedance variable part (E_block), electrical impedance fixed part (P_block) and mechanical (M_block).

3. The following parameters are detected in R_block:
a. The maximum amplitude value R (FIG. 6) peak is detected in R block between t0 and 80 ms:
i. Registration of R-peak time (tR) is performed;
b. Q-peak start time (tQ) is detected;
c. Minimum S-peak value (sA) and its time is detected (tS);
d. T-peak end time (tT) is detected;
e. J-point is detected after S-peak.

4. Detection of the wave (a) start is performed in E_block (FIG. 5):
a. The current wave is differentiated in E_block for the first time (differentiation period=0.001 s);
b. The time of the maximum value is detected in the differentiated wave (t2) (FIG. 4);
c. The maximum value of the first derivative amplitude is detected (A2);
d. The first row derivative obtained is differentiated again (differentiation period=0.001 s). The second row curve is obtained
e. The maximum value of the amplitude (dA2) and time (A2) are detected from the second row curve;
f. The second row derivative obtained is differentiated again (differentiation period=0.001 s). The third row curve is obtained;
g. The maximum value of the amplitude (dA3) and time (A3) are detected from the third row curve. This time is closest to the point (a).
Elimination of the breathing effect on the calculation results is performed, one of the possible ways of elimination is as follows: the wave amplitude value is registered in each wave at the point A3 (i), the regression line connecting this point with the detected point (A3 (i−1)) in the previous wave is formed and each newly detected line value is synchronically eliminated (subtracted) from the each wave value.

5. The following curve points are detected in E_block:
a. The point c at which the amplitude A5 and time (t3) are measured;
b. The point d at which the amplitude A6 and time (t4) are measured
c. The time (t5) is measured at the first derivative point e (systole end).

6. The amplitude value is registered in P_block at the time tA3 (Z₀, Om).

7. The following curve points are detected in M_block:
a. The amplitude value is registered at the time tA3 (OR, mmH₂O);
b. The variable pulse wave is obtained by subtracting this value from the whole M_block;
Elimination of the breathing effect on the calculation results is performed, one of the possible ways of elimination is as follows: the wave amplitude value is registered in each wave at the point tA3 (i), the regression line connecting this point with the point (tA3 (i−1)) detected in the previous wave is formed and each newly detected line value is synchronically eliminated (subtracted) from the each wave value;
c. Amplitudes M3 and M4 are measured in the variable pulse wave at times t3 and t4 detected from E_block.

8. The following parameters are calculated and collected in each block (i):
a. For the chronotropic heart function assessment:
i. Time interval between adjacent R ECG peaks (RR (i) interval=tR (i)−t R (i−1)), ms.

b. For the cardiac inotropic function assessment:
i. Pre-ejection period: PEP(i)=tA3−tQ−Delta, ms; Delta is a carotid pulse wave delay period that equals 18.5±8.2 ms.
ii. Left ventricular ejection time (systole): LVET(i)=t5−tA3, ms;
iii. Ratio of PEP and LVET: Ino(i)=PEP(i)/LVET(i).

c. For ventricular depolarization and repolarization state determination:
i. Detection of QT period: QT(i)=tT-tQ and assessment;
ii. ST-detection of depression levels from J-point to 80 ms after it (ST80(i), mikroV).

d. The following values are detected for arterial stiffness assessment:
i. Early systolic wave value, (ASB(i)=A5−A0);
ii. Late systolic wave value, (VSB(i)=A6−A0);
iii. Carotid augmentation index, CAIx(i)=100*(VSB−ASB)/ASB, percent;
iv. Maximum ejection force velocity value, dZ/dt(i)=A2;
v. Maximum ejection force value, d²Z/dt2 (i)=dA2;
vi. Pulse wave propagation retention period, PPT(i)=tA3−t2;
vii. Blood pulsing volume in carotids is calculated using a formula: CarVol(i), ml=rho*(L/Z₀)² LVET*A2, where L is the distance between electrodes, cm; Z₀ is fixed resistance value, om, rho=135 Om*cm.
viii. Carotid peripheral augmentation index: MPAIx(i)=100*(M4−M3)/M3, percent.
ix. Peripheral circulatory level variability: OR(i), mmH₂O.

Each pulse wave is differentiated three times. The maximum value of the first derivative (A2) indicates the maximum velocity of arterial filling, which depends on cardiac inotropy (contraction force) and arterial stiffness. The maximum value of the second derivative (dA2) indicates the acceleration (force) of the blood ejection from the heart, which is directly dependent on the maximum cardiac force used during the contraction. The third maximum value of the derivative (dA3) indicates the end of the cardiac period and the start of the next period.

If the analysis process takes longer (over 1 minute), then the dynamics analysis of parameters obtained is performed.

9. Statistical analysis of all collected parameters with a period of 0.5 minutes or more:
a. Averages, variance and standard deviation are calculated;
b. High frequency (0.15-0.4 Hz) variance (ms2) of the selected period of RR intervals is calculated.

10. The data analysis results obtained are summarized:
a. Cardiac chronotropic function assessment;
b. Cardiac inotropic function assessment;
c. Ventricular depolarization and repolarization status assessment;
d. Arterial stiffness assessment;
e. Comparison of results obtained over time.

The above listed calculation steps are implemented by electronic calculation means connected to parts of the equipment (FIG. 1), devices measuring physical parameters listed above, as well as having communication means with remote electronic calculation means. Said electronic calculation means being electronic devices having a processor (s) for processing data, temporary and/or permanent memory means for data and information storage, internal communication means for communication between structural elements and communication means with external devices. It can be a computer, a microcontroller equipped with special software for implementing calculation steps listed above. The electronic equipment implementing the analysis, calculation method may be remote from the part of the measurement equipment. Said equipment can be portable.

In order to illustrate and describe the invention, the description of the preferred embodiments is presented above. This is not a detailed or restrictive description to determine the exact form or embodiment. The above description should be viewed more than the illustration, not as a restriction. It is obvious that specialists in this field can have many modifications and variations. The embodiment is chosen and described in order to best understand the principles of the present invention and their best practical application for the various embodiments with different modifications suitable for a specific use or implementation adaptation. It is intended that the scope of the invention is defined by the definition added to it and its equivalents, in which all of these definitions have meaning within the broadest limits, unless otherwise stated.

In the embodiments described by those skilled in the art, modifications may be made without deviating from the scope of this invention as defined in the following definition.

1. A method for continuous monitoring over time of cardiac functions and vascular resistance by assessing changes in the carotid pulse wave form, carotid wave amplitude levels, cardiac contraction phases, heart rate variability with electrocardiogram, and measuring electrical tissue impedance,
wherein
the carotid pulse wave form parameters are measured
by an electrical impedance method when an electrical tissue resistance is measured; and
by a mechanical method when a change in the air pressure due to dilatation of blood vessel walls is measured in a closed cavity,
and the results of all measurements after synchronization under the electrocardiogram R-peak are processed and analysed to obtain the analysis results.

2. The method for continuous monitoring over time of cardiac functions and vascular resistance according to claim 1, wherein the following data processing steps are performed:
1. eliminating noise in three processes by filters without a phase shift (filtering twice up and down);
2. obtaining four process blocks during registration: ECG (R_block), electrical impedance variable part (E_block), electrical impedance fixed part (P_block) and mechanical (M_block);
3. detecting the following parameters in R_block:
a. The maximum amplitude value R (FIG. 6) peak is detected in R_block between tO and 80 ms:
i. Registration of R-peak time (tR) is performed;
b. Q-peak start time (tQ) is detected;
c. Minimum S-peak value (sA) and its time moment is detected (tS);
d. T-peak end time (tT) is detected;
e. J-point is detected after S-peak;
4. detecting the wave (a) start is performed in E_block:
a. The current wave is differentiated in E_block for the first time (differentiation period=0.001 s);
b. The time of the maximum value is detected in the differentiated wave (t2);
c. The maximum value of the first derivative amplitude is detected (A2);
d. The first row derivative obtained is differentiated again (differentiation period=0.001 s), the second row curve is obtained;
e. The maximum value of the amplitude (dA2) and time moment (A2) are detected from the second row curve;
f. The obtained second row derivative is differentiated again (differentiation period=0.001 s), the third row curve is obtained;
g. The maximum value of the amplitude (dA3) and time moment (A3) are detected from the third row curve; this time moment is closest to the point (a);
elimination of the breathing effect on the calculation results is performed, one of the possible ways of elimination is as follows: the wave amplitude value is registered in each wave at the point A3 (i), the regression line connecting this point with the point (A3 (i−1)) detected in the previous wave is formed and each newly detected line value is synchronically eliminated (subtracted) from the each wave value;
5. detecting the following curve points in E_block:
a. The point c at which the amplitude A5 and time (t3) are measured;
b. The point d at which the amplitude A6 and time (t4) are measured;
c. The time (t5) is measured at the first derivative point e (systole end);
6. The amplitude value is registered in P_block at the time tA3 (Z₀, Om);
7. The following curve points are detected in M_block:
a. The amplitude value is registered at the time tA3 (OR, mmH₂O);
b. The variable pulse wave is obtained by subtracting this value from the whole M_block;
elimination of the breathing effect on the calculation results is performed, one of the possible ways of elimination is as follows: the wave amplitude value is registered in each wave at the point tA3 (i), the regression line connecting this point with the point (tA3 (i−1)) detected in the previous wave is formed and each newly detected line value is synchronically eliminated (subtracted) from the each wave value;
c. Amplitudes M3 and M4 are measured in the variable pulse wave at times t3 and t4 detected from E_block;
8. The following parameters are calculated and collected in each block (i):
a. For the cardiac chronotropic function assessment:
i. Time interval between adjacent R ECG peaks (RR (i) interval=tR (i)−t R (i−1)), ms.;
b. For the cardiac inotropic function assessment is calculated:
i. Pre-ejection period: PEP(i)=tA3−tQ−Delta, ms; Delta is a carotid pulse wave delay period that equals 18.5±8.2 ms.;
ii. Left ventricular ejection time (systole): LVET(i)=t5−tA3, ms;
iii. Ratio of PEP and LVET: Ino(i)=PEP(i)/LVET(i);
c. For ventricular depolarization and repolarization status determination:
i. Detection of QT period: QT(i)=tT−tQ and assessment;
ii. ST-detection of depression levels from J-point to 80 ms after it (ST80(i), mikroV);
d. The following values are detected for arterial stiffness assessment:
i. Early systolic wave value, (ASB(i)=A5−A0);
ii. Late systolic wave value, (VSB(i)=A6−A0);
iii. Carotid augmentation index, CAIx(i)=100*(VSB−ASB)/ASB, percent;
iv. Maximum ejection force velocity value, dZ/dt(i)=A2;
v. Maximum ejection force value, d²Z/dt2 (i)=dA2;
vi. Pulse wave propagation retention period, PPT(i)=tA3−t2;
vii. Blood pulsing volume in carotids is calculated using a formula: CarVol(i), ml=rho*(L/Z₀)²*LVET*A2, where L is the distance between electrodes, cm; Z₀ is fixed resistance value, om, rho=135 Om*cm.;
viii. Carotid peripheral augmentation index: MPAIx(i)=100*(M4−M3)/M3, percent;
ix. Peripheral circulatory level variability: OR(i), mmH₂O;
9. Statistical analysis of all collected parameters with a period of 0.5 minutes or more:
a. Averages, variance and standard deviation are calculated;
b. High frequency (0.15-0.4 Hz) variance (ms2) of the selected period of RR intervals is calculated;
10. The data analysis results are summarized:
a. Cardiac chronotropic function assessment;
b. Cardiac inotropic function assessment;
c. Ventricular depolarization and repolarization status assessment;
d. Arterial stiffness assessment;
e. Comparison of results obtained over time.

3. An equipment for continuous monitoring over time of cardiac functions and vascular resistance, implementing the method according to claim 1.

4. The equipment for continuous monitoring over time of cardiac functions and vascular resistance according to claim 3, wherein the air pressure in ear canals and the electrical head tissue impedance is measured by a device with two plugs fitted into ear canals for measurement, each plug has an electrical impedance current electrode and measurement electrode separated by an electrical insulating material.

5. The equipment for continuous monitoring over time of cardiac functions and vascular resistance according to claim 4, wherein the air pressure in ear canals and the electrical head tissue impedance is measured by a device with two plugs fitted into ear canals for measurement, each plug has an air channel to a sensor measuring the air pressure.

6. The equipment for continuous monitoring over time of cardiac functions and vascular resistance according to claim 5, wherein the air pressure in ear canals and the electrical head tissue impedance is measured by a device with two plugs fitted into ear canals for measurement, each channel connecting the plug and the sensor has air flow closing means, such as a valve.

7. The equipment for continuous monitoring over time of cardiac functions and vascular resistance according to claim 4, wherein the air pressure in ear canals and the electrical head tissue impedance is measured by a device with two plugs fitted into ear canals for measurement, each plug has an air pressure measurement sensor.

8. The equipment for continuous monitoring over time of cardiac functions and vascular resistance according to claim 3, wherein the air pressure in ear canals is measured by a device with two plugs fitted into ear canals for measurement, each plug has an air pressure measurement sensor and the electrical head tissue impedance is measured by a device with a current electrode pressed on one side of the earlobe, and the measurement electrode pressed on the other (opposite) side of the earlobe.

9. The equipment for continuous monitoring over time of cardiac functions and vascular resistance according to claim 3, wherein the air pressure in ear canals is measured by a device having two plugs, which are fitted into ear canals for measurement, each plug has an air pressure measurement sensor and the electrical head tissue impedance is measured by a device with an electrical impedance measurement current electrode attached to the skin behind the ear at the nipple (mastoid) bones and a measurement electrode near the current electrode, one pair of electrodes on one side of the head, the other pair of electrodes on the other side of the head.
